(19) United States
(12) Patent Application Publication
(10) Pub. No.: US 2022/0305244 A1
(43) Pub. Date: Sep. 29, 2022

(54) Multi-Functional Integrated Skin Beauty Device

(71) Applicant: SOOVON Co., Ltd., Incheon (KR)

(72) Inventor: Kyoung Hee OH, Goyang-si (KR)

(21) Appl. No.: 17/231,300

(22) Filed: Apr. 15, 2021

(30) Foreign Application Priority Data
Mar. 25, 2021 (KR) 10-2021-0039047

(51) Int. Cl. A61M 35/00; C02F 1/461; C02F 1/467; A61M 11/00; A61N 1/04; A61N 1/32; A61N 5/06; A61F 7/00; A61L 2/22; B05B 9/01; B05B 17/06

(52) U.S. Cl. CPC A61M 35/003; C02F 1/4618; C02F 1/4676; A61M 11/005; A61N 1/0408; A61N 1/328; A61N 5/0616; A61F 7/00; A61L 2/22; B05B 9/01; B05B 17/0607; C02F 2001/4619; C02F 2103/026

(57) ABSTRACT

A multi-functional integrated skin beauty device according to the present invention is formed by comprising a mist treatment part generating sterilization water by electrolyzing water in a water tank and spraying the generated sterilization water in a mist shape, a temperature stimulation treatment part applying a temperature stimulation to skin, an EP treatment part applying an electric signal to an EP electrode; and a light treatment part outputting a light for skin care. A housing forming an exterior appearance of the multi-functional integrated skin beauty device includes a pillar-type handle part graspable by a user with one hand, and a first head part and a second head part protruded from an upper end of the handle part toward both lateral surfaces.

### CROSS-REFERENCE TO RELATED APPLICATION

This application claims priority to and the benefit of Korean Patent Application No. 10-2021-0039047 filed on Mar. 25, 2021 in the Korean Intellectual Property Office, the entire content of which is incorporated herein by reference.

### TECHNICAL FIELD

The teachings in accordance with exemplary and non-limiting embodiments of this invention relate generally to a portable beauty device, and more particularly, to a multi-functional integrated skin beauty device conveniently useable by being effectively integrated with multiple functions including a spraying/atomizing function of hydrogen sterilization water in mist by generating the hydrogen sterilization water through electrolysis, a temperature stimulation function for skin care, an electroporation function, an optical irradiation function, a massage function, and an oil/water measurement function.

### BACKGROUND OF THE INVENTION

Recently, women as well as men are particularly concerned about their beauty in order to preserve a beautiful image and appearance. In general, various cosmetics are widely used for beauty treatment such as moisturizing, nutrition supply, collagen that helps activate the skin function, nutritional fluid and essence.

Furthermore, mist suppliers are being used for skin care, where the mist suppliers spout mist of fine particulate state.

The mist supplier may be used for various purposes including, but not limited to, moisturizing, whitening effect, nourishment and the like, and in particular, may be used for multipurpose of skin cleanness.

The laid-open patent No.: 10-2020-0086433 discloses a portable mist device that generates hydrogen sterilization water using electrolysis, and spouts the generated hydrogen sterilization water in mist shape through ultrasonic oscillator to thereby be conducive to skin care.

Meantime, skin care may require various elements including not only mist but also temperature stimulation, EP (Electropolation) stimulation, optical stimulation and physical massage through vibration.

For example, the Korean registered patent No.: 10-1551923 discloses a hydrogen cosmetics penetration supply device using an electroporation and the Korean registered patent No.: 10-2030528 discloses a skin care device generating plasma and electroporation operation to skin. In addition, the temperature stimulation including cold stimulation and warm stimulation adequately applied to skin may provide a desirable beneficial effect to skin care.

However, the disadvantage is that the aforementioned conventional skin care functions cannot be effectively fused together with the hydrogen sterilization water mist function capable of performing to maintain a clean skin.

When the hydrogen sterilization water mist function, which is the most fundamental base for skin care, is not efficiently fused with various functions used for skin care including an EP stimulation, an optical stimulation, and a temperature stimulation, users are forced to use and prepare two or more devices individually to thereby increase an economic burden and to make it difficult to maintain/repair the devices, whereby the skin care may become discomfortable.

### PRIOR TECHNICAL DOCUMENTS

### Patent Documents

(1) The Korean Laid-Open Patent No.: 10-2020-0086433 (Laid-open on Jul. 17, 2020)
(2) The Korean Registered Patent No.: 10-1551923 (Published Date: Sep. 10, 2015)
(3) The Korean Registered Patent No.: 10-2030528 (Published Date: Nov. 8, 2019)

### SUMMARY OF THE INVENTION

### Technical Subject

The present invention is devised to solve the aforementioned disadvantages/problems and it is an object of the present invention to provide a multi-functional integrated skin beauty device configured to be realized on a portable device and further improved in use convenience, which is fundamentally equipped with a spraying function of sterilization water in mist using electrolysis, and along with a temperature stimulation function, an electroporation function, an optical stimulation function, a massage function, and an oil/water measurement function.

### Technical Solution

In one general aspect of the present invention, there is provided a multi-functional integrated skin beauty device, the device comprising:

a mist treatment part generating sterilization water by electrolyzing water in a water tank and spraying the generated sterilization water in a mist shape;

a temperature stimulation treatment part applying a temperature stimulation to skin using an thermoelement;

a power supply part supplying an electric power;

an input part receiving an operation command; and

a controller controlling the mist treatment part and the temperature stimulation treatment part in response to the operation command inputted through the input part.

Preferably, but not necessarily, a housing forming an exterior appearance of the multi-functional integrated skin beauty device may include:

a pillar-type handle part graspable by a user with one hand; and

a first head part protruded from an upper end of the handle part to a lateral surface and a second head part protruded to an opposite lateral surface.

Preferably, but not necessarily, the first head part may be disposed with a raw water inlet to allow raw water to be poured into a water tank of the mist treatment part and the second head part may be disposed with a skin contact member to allow applying a temperature stimulation by being contacted with skin.

Preferably, but not necessarily, the multi-functional integrated skin beauty device may further comprise:

an EP treatment part applying an electric signal to an EP electrode; and

a light treatment part outputting a skin care light.

Preferably, but not necessarily, the second head part may be disposed with a transparent window formed at a surrounding of the skin contact member to output a light for skin care.

Preferably, but not necessarily, the skin contact member and an outlying electrode member formed along a surrounding of the transparent window may form the EP electrode.

Preferably, but not necessarily, an uppermost end of the second head part may be disposed higher than the first head part.

Preferably, but not necessarily, the mist treatment part may include:
an electrode module disposed inside of the water tank to transform the raw water to sterilization water by electrolyzing the raw water;

an ultrasonic oscillator generating a fine particulate type mist by applying a vibration to the sterilization water generated by the electrode module; and

a mist outlet to allow spouting the mist generated by the ultrasonic oscillator therethrough.

Preferably, but not necessarily, the water tank may be formed of an approximately ‘¬’ shape across an upper space of the handle part from the raw water inlet.

Preferably, but not necessarily, at least a portion of the electrode module may be so disposed at a place as to be visible through the raw water inlet.

Preferably, but not necessarily, the mist outlet may be disposed at an upper end of the handle part toward a direction formed with the raw water inlet, and the ultrasonic oscillator may be disposed at a surrounding of the mist outlet. At this time, a floor position of the water tank may be so disposed as to be adjacent to a lowermost position of the ultrasonic oscillator.

Preferably, but not necessarily, the multi-functional integrated skin beauty device may further comprise:

an inlet cap configured to open/close the raw water inlet;

a visual effect plate configured to display a visual effect by being so disposed at an inside of the water tank as to allow being visible from the raw water inlet; and

an illumination part configured to shed an illumination light into an inside of the water tank from under the floor of the water tank.

Preferably, but not necessarily, at least a portion of the water tank may be so disposed with a transparent or translucent material as to allow the illumination light coming off from the illumination part to pass therethrough.

Preferably, but not necessarily, at least a portion of the inlet cap may be so disposed as to allow an interior of the water tank to look enlarged.

Preferably, but not necessarily, the multi-functional integrated skin beauty device may further comprise a cradle part so disposed with a battery charging terminal as to allow a lower end of the handle part to rest thereon.

Preferably, but not necessarily, the cradle part may be disposed at a floor with a water drain hole to allow the water flowing down through the handle part to be drained out.

Preferably, but not necessarily, a portion formed with the water drain hole may be formed lower than a portion disposed with the battery charging terminal.

Preferably, but not necessarily, the thermoelement forming the temperature stimulation treatment part may be formed by including a peltier device. At this time, one surface of the peltier device may be so disposed as to thermally contact the water stored in the water tank of the mist treatment part.

Preferably, but not necessarily, the thermal contact may be realized through a first heat dissipation part of first material directly contacting the water stored in the water tank of the mist treatment part and a second heat dissipation part of second material interposed between one surface of the peltier device and the first heat dissipation part.

Preferably, but not necessarily, the first material may be formed with a material less contaminated than the second material by the sterilization water generated through the electrolysis. For example, the first material may include an SUS material and the second material may include an aluminum material.

Preferably, but not necessarily, the second heat dissipation part may be so disposed as to accommodate a cooling liquid at an inside thereof.

Preferably, but not necessarily, the second heat dissipation part may be so disposed as to tight-seal the cooling liquid by closing a lid after the cooling liquid is poured into an inner space thereof.

Preferably, but not necessarily, the thermal contact between the first heat dissipation part and the second heat dissipation part may be realized at an area where the water tank is curbed.

### Advantageous Effects

According to the present invention, a user can care skin more simply and more efficiently because the user can utilize not only the hydrogen sterilization water spraying/atomizing function but also the temperature stimulation function, the electroporation function, the light stimulation function, the massage function, and the oil/water measurement function using a single portable device.

Particularly, when the temperature stimulation function, the electroporation function, the light stimulation function, the massage function, and the oil/water measurement function are intensively and collectively performed on one surface contacted by the skin, the skin care can be more effectively realized.

Furthermore, the multi-functional integrated skin beauty device can maximize a mist spraying efficiency by optimizing the structure of a water tank, increase a user satisfaction by realizing various visual effects and further enhance the safety of the water-using device through the water drainage function of cradle part.

### BRIEF DESCRIPTION OF THE DRAWINGS

FIG. 1 is a functional block diagram illustrating a multi-functional integrated skin beauty device according to an exemplary embodiment of the present invention.

FIG. 2 is an example explaining that a temperature stimulation function, an electroporation function and a light stimulation function are intensively and collectively realized on one surface contacted by the skin.

FIGS. 3, 4 and 5 are examples of a housing portably forming a multi-functional integrated skin beauty device according to the present invention.

FIG. 6 is an example illustrating a mist treatment part and a temperature stimulation part each disposed on both sides of a head part of a housing.

FIG. 7 illustrates a mist treatment part according to a detailed exemplary embodiment of the present invention.

FIG. 8 illustrates an upper cross-sectional surface of a housing according to an exemplary embodiment of the present invention.

FIG. 9 illustrates arrangement of skin contact member, transparent window and electroporation electrode according to a detailed exemplary embodiment of the present invention.

FIG. 10 illustrates a temperature stimulation treatment part according to a detailed exemplary embodiment of the present invention.

FIG. 11 illustrates a first heat dissipation part directly contacting sterilization water inside a water tank according to an exemplary embodiment of the present invention.

FIG. 12 illustrates a structure of second heat dissipation part according to a detailed exemplary embodiment of the present invention.

FIG. 13 illustrates an inner appearance of a water tank and illumination effect by allowing an inlet cap to be transparently formed according to a detailed exemplary embodiment of the present invention.

FIG. 14 illustrates a cradle part according to an exemplary embodiment of the present invention.

FIG. 15 illustrates an example of a cross-section of a cradle part.

### DETAIL DESCRIPTIONS OF THE INVENTION

The present invention may be applied with various changes and have several exemplary embodiments, where particular exemplary embodiments will be exemplified in the drawings and described in detail through the detailed description of the present invention. However, it should be understood that the present invention is not limited to particular embodiments, but encompasses all changes, modifications, equivalents and substitutes included within the ideas and technical scopes of the present invention.

In describing the present invention, detailed descriptions of well-known technologies are omitted for brevity and clarity so as not to obscure the description of the present invention with unnecessary detail.

The terminology used herein is for the purpose of describing exemplary embodiments only and is not intended to be limiting. As used herein, the singular forms may be intended to include the plural forms as well, unless the context clearly indicates otherwise.

It will be further understood that the terms “comprises” and/or “comprising,” or “includes” and/or “including” when used in this specification, specify the presence of stated features, regions, integers, steps, operations, elements, and/or components, but do not preclude the presence or addition of one or more other features, regions, integers, steps, operations, elements, components, and/or groups thereof.

It will be understood that, although the terms first, second, etc. may be used herein to describe various elements, these elements should not be limited by these terms. These terms are only used to distinguish one element from another.

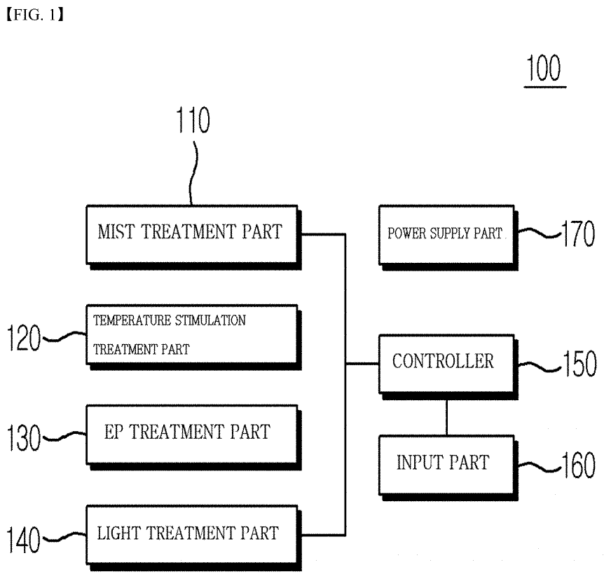

FIG. 1 is a functional block diagram illustrating a multi-functional integrated skin beauty device 100 according to an exemplary embodiment of the present invention, where the device 100 may be formed by including a mist treatment part 100 generating a sterilization water by electrolyzing the water inside a water tank and spraying the generated sterilization water in a mist shape, and a temperature stimulation treatment part 120 applying a temperature stimulation to a user skin by using a thermoelement, and the device 100 may be formed by further including an EP treatment part 130 applying an electric signal to an EP electrode, and a light (optical) treatment part 140 outputting a skin care light capable of being used for skin care. Furthermore, the device 100 may further include a vibration part providing a physical massage and an oil/water measurement part.

Furthermore, the multi-functional integrated skin beauty device 100 may be formed by including an input part 160 receiving an operation command from a user, and a controller 150 controlling the overall operations of the multi-functional integrated skin beauty device 100 including the mist treatment part 110, the temperature stimulation treatment part 120, the EP treatment part 130 and the light treatment part 140 in response to the operation command inputted through the input part 160, and a power supply part 170 supplying an electric power to the device 100.

The input part 160 may receive various operation commands on the multi-functional integrated skin beauty device 100 from a user. The operation commands received from the input part 160 and input methods may be variably configured, and the present invention is not particularly limited thereto.

For example, the input part 160 may include various switches and buttons related to a variety of functions, including but not limited to, power ON/OFF of the multi-functional integrated skin beauty device 100, mist function ON/OFF, temperature stimulation function ON/OFF, EP function ON/OFF, light stimulation ON/OFF, light type set-up function, cold stimulation set-up, warm stimulation set-up, temperature set-up, massage intensity control function, oil/water measurement and control and the like.

The EP treatment part 130 may increase the penetrability of skin cell membrane by applying an electric signal to an electroporation electrode to thereby add an electric field to the skin cell membrane of a user.

The voltages, waveforms and frequencies of the electric signal applied to the electroporation electrode by the EP treatment part 130 may be variably configured according to the need, and the shape, structure and placement of the electroporation electrode may be also variably formed.

The light treatment part 140 may output one or more types of skin-care lights useable for skin care, and may, simultaneously or selectively, output lights of various wavelength ranges (e.g.: 400-470 nm, 570-590 nm, 630-700 nm, 800-1,200 nm, etc.).

The light treatment part 140 may output skin-care lights through an LED (Light-Emitting Diode), and the wavelength, intensity and pattern of lights may be variably organized.

The lights outputted from the light treatment part 140 may show various skin-care effects, including but not limited to, antioxidation, whitening, skin tone-up, wrinkle recovery, sensitive skin soothing, acne improvement, blemishes/pigmentation control, skin soothing, strengthening skin elasticity, improving blood circulation, pore contraction, inhibition of aging, skin regeneration and the like, depending on characteristics thereof.

The temperature stimulation structure for temperature stimulation, the electroporation electrode structure for electroporation stimulation and light output structure for light stimulation and the like may be variably organized.

Particularly, the multi-functional integrated skin beauty device 100 according to an exemplary embodiment of the present invention may be so configured as to be intensively provided on one surface contacted by a user skin with temperature stimulation function, electroporation function and light stimulation function, whereby the skin care effect can be more efficiently improved.

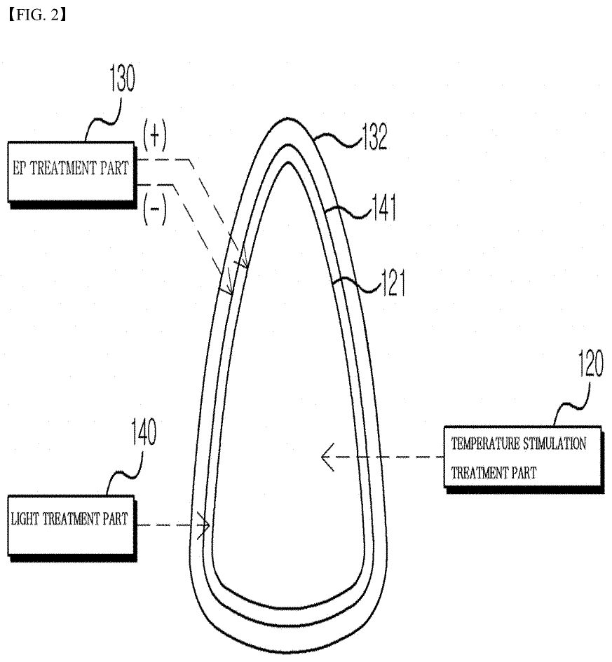

Referring to FIG. 2, the temperature stimulation treatment part 120 may perform the role of applying a temperature stimulation to a user skin by using a thermoelement, and may be formed by including a skin contact member 121 which is an element heated or cooled by being directly contacted to a user skin.

The skin contact member 121 may be disposed on one surface contacted by a user skin at a housing of the multi-functional integrated skin beauty device 100.

Furthermore, a transparent window 141 may be disposed along a surrounding of the skin contact member 121 to allow a light outputted from the light treatment part 140 to be emitted, and an outlying electrode member 132 may be disposed along an outer surrounding of the transparent window 141.

At this time, the skin contact member 121 and the outlying electrode member 132 may be formed with an electrically conductive material, and the skin contact member 121 and the outlying electrode member 132 may form the electroporation electrode.

That is, the skin contact member 121 may be a positive pole (+) or negative pole (−) electrode for electroporation stimulation in addition to the role of applying a temperature stimulation to the skin, and the outlying electrode member 132 may be an electrode having an opposite polarity (positive pole or negative pole) of the skin contact member 121.

Although FIG. 2 has illustrated examples in which each of the skin contact member 121, the transparent window 141 and the outlying electrode member 132 takes approximately a triangular shape, it should be apparent that material, shape and placement or arrangement of the skin contact member 121, the transparent window 141 and the outlying electrode member 132 may be variably organized, and the present invention is not limited thereto. For example, it should be apparent that the skin contact member 121, the transparent window 141 and the outlying electrode member 132 may take an arbitrary shape including a round shape, a polygonal shape including a square shape or an oval shape, and each may not take the same shape.

As one of the embodying methods to allow each function to be intensively and integrally provided on one surface, and each function to cause an upward effect on the other, the transparent window 141 may be formed in a ring shape encompassing an outlying area of the skin contact member 121. Furthermore, the outlying electrode member 132 formed along an outer surrounding of the transparent window 141 may be formed in a ring shape encompassing the transparent window 141.

Although a housing forming an outer appearance of the multi-functional integrated skin beauty device 100 may be variably formed as diverse as possible, it is preferable that the housing be formed with a portable shape to allow a user to use by grasping with one hand.

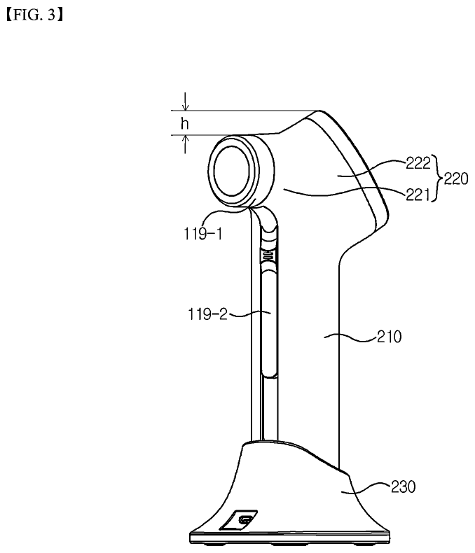

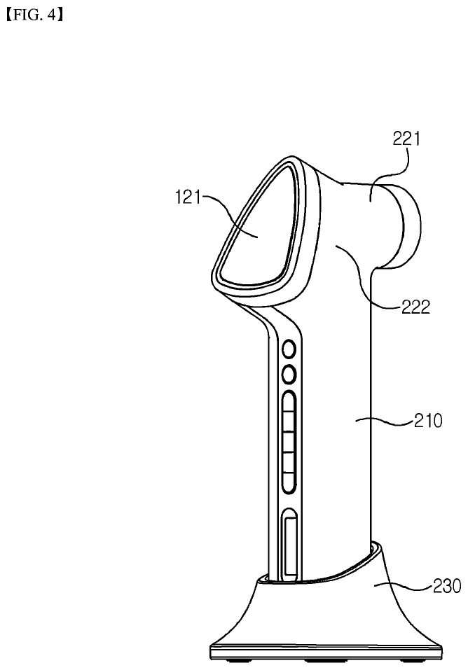

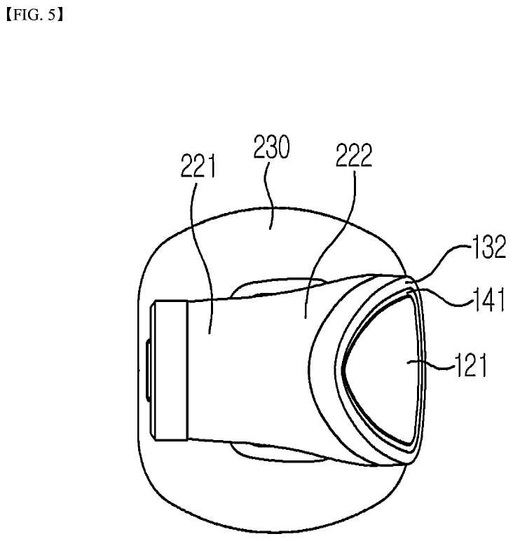

FIGS. 3, 4 and 5 illustrate an example of a housing portably forming the multi-functional integrated skin beauty device 100, where FIG. 3 is a perspective view seen from a direction from which mist is spouted, FIG. 4 is a perspective view seen from a direction where the skin contact member 121 contacts a user skin in order to provide a temperature stimulation, and FIG. 5 is a view seen from a top of a housing.

The housing may be formed by including a pillar-shaped handle part 210 to allow a user to grasp with one hand, and a head part 220 protruded from an upper end of the handle part 210 toward both lateral surfaces thereof. At this time, the head part 220 may include a first head part 221 protruded from an upper end of the handle part 210 to one lateral surface and a second head part 222 protruded toward an opposite lateral surface thereof.

The position (height) and angle protruded by the first head part 221 and the second head part 222 from the handle part 210 may be variably implemented. Furthermore, the shape of the first head part 221 and the second head part 222 may be also variably implemented.

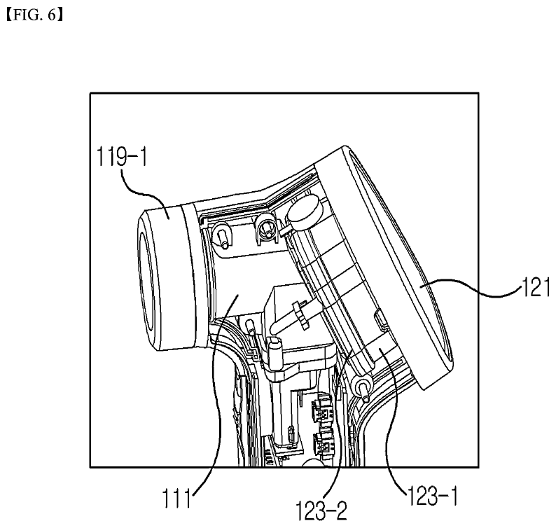

When referred along with FIG. 6, the first head part 221 of the housing may be disposed with various elements for mist treatment, and the second head part 222 protruded from the other lateral surface may be disposed with other elements for temperature stimulation treatment.

A distal end surface of the first head part 221 may be formed with a raw water inlet to allow the water to be poured into a water tank 111 of the mist treatment part 110, and a distal end surface 3 of the second head part 222 may be disposed with a skin contact member 121 applying a temperature stimulation.

That is, the first head part 221 may be related to raw water supply for generation of mist and water tank space arrangement, and the second head part 222 may be a portion that applies stimulation by being contacted to a user skin.

Meantime, when the first head part 221 is disposed at the same height as that of the second head part 222 or disposed at a height higher than that of the first head part 221, there may be generated a discomfort in the course of using the second head part 222.

For example, the process of a user stimulating a facial skin using the second head part 222 may be disturbed because of the first head part 221 being hitched at the bridge of a nose.

In relation thereto, as shown in FIG. 3, an uppermost end of the second head part 222 may be disposed at a height higher than that of the first head part 221 at a predetermined length (h).

Then, the discomfort that may be generated by the first head part 221 may be prevented when the temperature stimulation, the electroporation stimulation and light stimulation are applied to the skin through the distal end surface of the second head part 222. When the distal end surface of the second head part 222 is formed with a triangular shape as shown in the figure, there may be an advantage of the process being more convenient where a facial skin is cared including a nosal area using the second head part 222.

Furthermore, the size of the distal end surface of the second head part 222 is formed larger than that of the first head part 221, various functions implemented through the distal end surface of second head part 222 may become more conveniently useable. Although the given figures illustrate that the second head part 222 first starts to protrude from the handle part 210 ahead of the first head part 221 to thereby be formed with a broader size up to a position higher than the first head part 221, the present invention is not limited thereto.

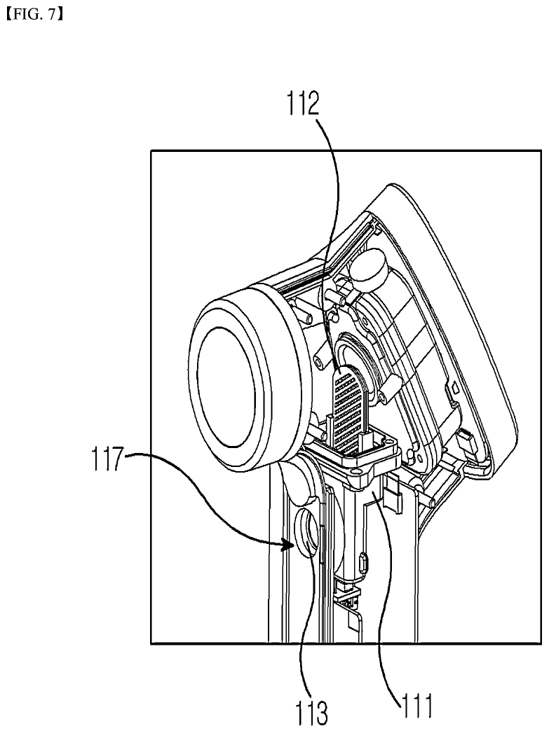

Referring to FIGS. 6 and 7, the mist treatment part 110 may be formed by including a water tank 111 holding the raw water including the faucet water, an electrode module 112 disposed inside the water tank 111 to transform the raw water to sterilization water through electrolysis, an ultrasonic oscillator 113 generating a mist of fine particulate shape by applying vibration to the sterilization water generated by the electrode module 112, and a mist outlet 117 to allow the mist generated from the ultrasonic oscillator 113 to be ejected therethrough.

The structure, shape and material of water tank 111 may be variably organized as necessary, and the present invention is not particularly limited thereto.

A distal end surface of first head part 221 may be formed with a raw water inlet to allow the water to be poured into the water tank 111 of the mist treatment part 110, where the water tank 111 may be formed of an approximately shape across an upper space of the handle part 210 from the raw water inlet.

The raw water inlet formed at the distal end surface of first head part 221 may be opened/closed through an inlet cap 119-1, and a user may open the inlet cap 119-1, pour the raw water into the water tank 111, close the inlet cap 119-1 and tight-seal the raw water inlet.

Furthermore, an upper end of the handle part 210 may be disposed with a mist outlet 117 toward a direction where the raw water inlet is formed.

Each of the electrode modules 112 are formed to be spaced apart from the other, and may be formed by including a pair or more of electrode plates each connected to a negative pole (−) and a positive pole (+), and dissolve the water molecules using the principle of underwater discharge, whereby the sterilization water having the sterilizing power can be generated.

That is, the sterilizing power can be possessed by allowing various negative ions (O⁻, O₃⁻, OH⁻, HOCl, H₂O₂) to be generated because plasma discharge is caused in the underwater through two electrode plates. The electrode modules 112 may be variably formed.

The ultrasonic oscillator 113 may be so disposed as to encompass a surrounding of the mist outlet 117 to efficiently spout the mist.

As one example, when the mist outlet 117 is formed to be round, the ultrasonic oscillator 113 may be formed with a ring shape to encompass a surrounding of the mist outlet 117.

With reference to mist generation using the ultrasonic oscillator 113, the shape of the water tank 111 at an area where the ultrasonic oscillator 113 is disposed may be formed with a front and rear length as narrow as possible and with a left and right length to be similar to an area of the ultrasonic oscillator 113.

Here, the front and rear length means a length between a surface where the ultrasonic oscillator 113 is disposed and an opposite surface thereto. Then, the mist ejection efficiency through the ultrasonic oscillator 113 can be enhanced.

For example, when the sterilization water is insufficient, a sufficient sterilization water can be supplied to the ultrasonic oscillator 113 for as long as possible. Because the sterilization water can be spread at an ultrasonic oscillator-disposed area as long as the water tank 111 is broad, an area can be reduced where the ultrasonic oscillator 113 and the sterilization water are contacted.

Furthermore, because the sterilization water contained at a position lower than the lowermost position of the ultrasonic oscillator 113 has a difficulty in being transformed to mist, the floor position of the water tank 111 may be so disposed as to be adjacent to the lowermost position of the ultrasonic oscillator 113.

As a result, an area contacted by the sterilization water and the ultrasonic oscillator 113 can be broadly maintained for as long as possible in the course of the sterilization water being reduced.

When a power is supplied to the electrode module 112 in response to the control of the controller 150, the electrolysis is performed to allow the raw water to be transformed to sterilization water, and the sterilization water mist is generated by the ultrasonic oscillator 113 to be spouted through the mist outlet 117.

FIG. 3 illustrates an example of a configuration where an ON/OFF switch for mist function is realized by an UP/DOWN sliding button 119-2 disposed underneath the mist outlet 117.

Although the electrode module 112 of mist treatment part 110 may be disposed at an arbitrary position inside the water tank 111, at least a portion of the electrode module 112 may be so disposed as to allow being looked through the raw water inlet, as an example related to visible effect.

Then, a user may visually ascertain an image of the sterilization water being generated by the electrode module 112 through the raw water inlet.

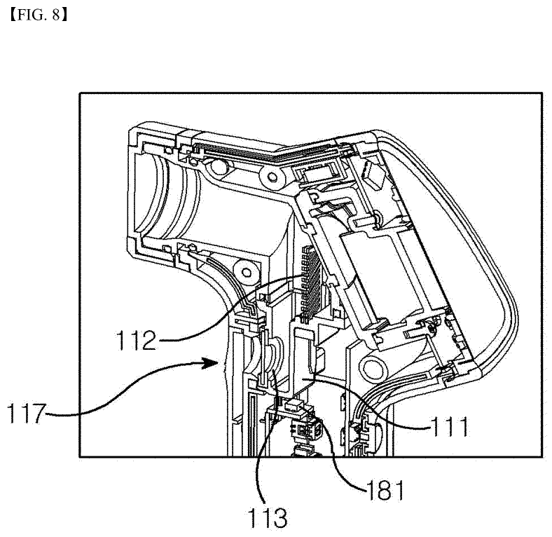

Referring to FIG. 8, the multi-functional integrated skin beauty device 100 may be formed by including an inlet cap 119-1 configured to open/close the raw water inlet, a visual effect plate (not shown) configured to display a visual effect by being so disposed at an inside of the water tank 111 as to allow being visible from the raw water inlet; and an illumination part 181 configured to shed an illumination light into an inside of the water tank 111 from under the floor of the water tank 111, wherein at least a portion of the water tank 111 is so disposed with a transparent or translucent material as to allow the illumination light coming off from the illumination part 181 to pass therethrough.

The visual effect seen from the raw water inlet may become more interesting due to the illumination light coming off through from the illumination part 181.

The illumination part 181 may be variably organized in order to shed the illumination light into the water tank 111. One of the examples is that the illumination part 181 may be formed using one or more LEDs but the present invention is not limited thereto.

The visual effect plate (not shown) is a plated element disposed to provide a visual effect, and material, structure, size, shape and arrangement of the visual effect plate may be variably implemented.

For example, the visual effect plate may be displayed with various types of information and advertisements including characters, figures and images, and may be formed with material capable of demonstrating a special visual effect when a light is incident. However, the present invention is not limited thereto.

The visual effect plate may display a visual effect by being so disposed at an inside of the water tank 111 as to allow being visible from the raw water inlet.

The visual effect plate may be disposed at variable positions. As a detailed example, the visual effect plate may be disposed at an area where the water tank is curbed, at a front side (a side where the raw water inlet is situated) of electrode module 112, a rear side or an upper side of the electrode module 112. However, the present invention is not limited thereto.

When the visual effect plate is arranged, a user can see not only an image of the sterilization water being generated through the raw water inlet, and an inside image of the water tank seen in a subtle light by the illumination light but also various special effects including various types of information and holograms.

Furthermore, at least a portion of the inlet cap 119-1 may be so disposed as to allow an interior of the water tank 111 to look enlarged.

That is, the inlet cap 119-1 may be so disposed transparently or translucently as to allow a user to see an interior of the water tank 111 even when the raw water inlet is closed.

Furthermore, the inlet cap 119-1 may be so disposed as to allow an interior of the water tank 111 to look enlarged like a convex lens or reading glasses.

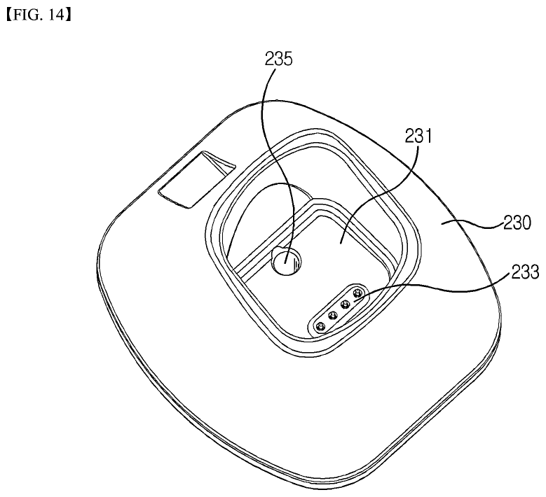

FIG. 13 illustrates a multi-functional integrated skin beauty device 100 according to a detailed exemplary embodiment of the present invention, and shows an example of an inner portion of the water tank 111 seen through a transparent reading glass material 119-3 while the inlet cap 119-1 is closed.

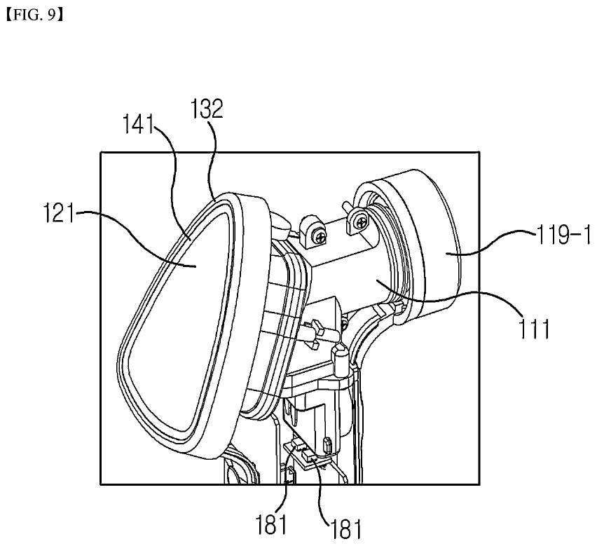

FIG. 9 illustrates the temperature stimulation function, the electroporation function and the light stimulation function being intensively provided on a distal end surface of the second head part 222 according to a detailed exemplary embodiment of the present invention.

The shape, structure and material of skin contact member 121 may be variably implemented.

For example, the skin contact member 121 may be formed with a material that enables an electric signal to be applied in order to have adequate heat transfer properties and to provide electroporation stimulation as well. Although the figure shows an example of skin contact member 121 formed with a triangular plated shape, the present invention is not limited thereto.

A transparent window 141 may be disposed along a surrounding of the skin contact member 121 in order to emit a light outputted from the light treatment part 140, and one or more of the LED elements forming the light treatment part 140 may be disposed at an inside of the transparent window 141. Furthermore, an outlying electrode member 132 may be disposed along an outer surrounding of the transparent window 141.

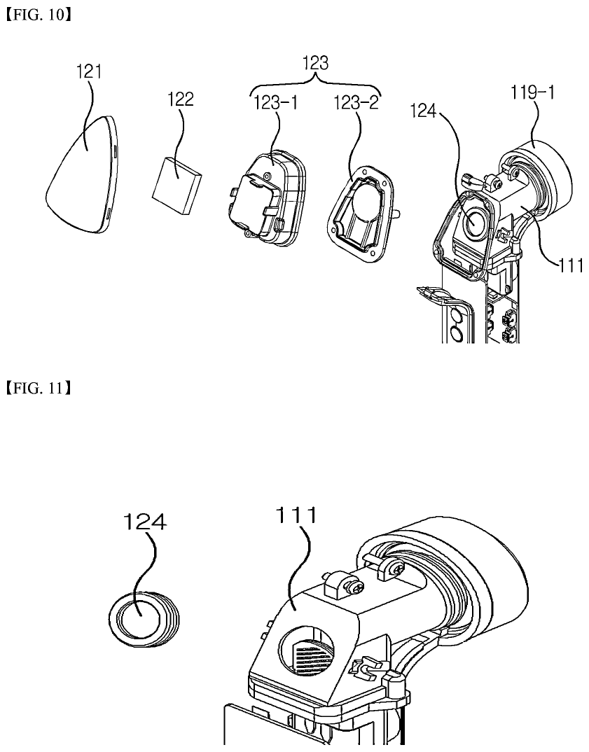

Referring to FIG. 10, the temperature stimulation treatment part 120 may be formed by including a skin contact member 121 applying a temperature stimulation by being contacted to a user skin, and a thermoelement 122 disposed to heat or cool the skin contact member 121.

The thermoelement 122 for temperature adjustment may be variably formed, and particularly formed using a peltier element.

The role of the skin contact member 121 may be so formed as to allow one surface of the peltier element 122 to directly perform.

One surface (external surface) of the peltier element 121 contacted by the skin contact member 121 may be treated with a particular temperature capable of stimulating the user skin. That is, when a current is applied to the peltier element 122 in response to the control of the controller 150, a relevant surface of the peltier element may be heated or cooled in response to the direction of the applied current.

Particularly, an inner surface of the peltier element 122 must be adequately cooled in order to perform a stable operation including protection of the peltier element.

Various methods for the said cooling may be utilized, and particularly, the inner surfaced of the peltier element 122 may be so disposed as to allow thermally contacting the water stored in the water tank 111 of the mist treatment part 110.

This means that the sterilization water stored in the water tank 111 of the mist treatment part 110 may be used for cooling of the peltier element 122.

The thermal contact between the inner surface of the peltier element 122 and the water stored in the water tank 111 of the mist treatment part 110 may be realized through a first heat dissipation part 124 of first material directly contacting the water stored in the water tank 111 and a second heat dissipation part 123 of second material disposed between the inner surface of the peltier element 122 and the first heat dissipation part 124.

That is, the water stored on the inner surface of the peltier element 122 and the water tank 111 may be thermally contacted by at least three mutually different elements.

Referring to FIG. 11, the first heat dissipation part 124 installed through a hole formed on the water tank 111 may contact, at one surface, the sterilization water inside of the water tank 111, and simultaneously contact the second heat dissipation part 123 at the other surface, where a portion installed with the first heat dissipation part 124 must be airtightly sealed by an O-ring to prevent the sterilization water from being leaked.

The first heat dissipation part 124 may be disposed at an external side (water tank side) of the second heat dissipation part 123 (see FIG. 10). The first heat dissipation part 124 may maximize the heat dissipation effect by being contacted with the sterilization water inside the water tank 111.

The shape of the first heat dissipation part 124 may not be particularly limited and may be variably formed such that the first heat dissipation part 124 may take a round shape, a polygonal shape and/or a star shape. When the first heat dissipation part 124 is disposed at an inner surface or at an outer surface with a plurality of lugs, the heat dissipation effect can be further enhanced.

Furthermore, a surface contacted by the first heat dissipation part 124 with the sterilization water of the water tank 111 may have the possibility of being contaminated by various ions and the like. Therefore, the first material may be formed with a material less contaminated by the sterilization water than the second material.

As a detailed example, the first material of first heat dissipation part 124 may be formed by including an SUS material and the second material of the second heat dissipation part 123 may be formed by including an aluminum material.

In this case, the surface contacted by the sterilization water of the water tank 111 may be minimized in oxidation.

As shown in FIG. 10, the thermal contact between the first heat dissipation part 124 and the second heat dissipation part 123 may be realized at an area where the water tank is curbed.

Meantime, the second heat dissipation part 123 may be formed by a water-cooling system that accommodates a cooling liquid therein in order to further improve the heat dissipation function. At this time, the second heat dissipation part 123 may be formed by including a main body 123-1 accommodating a cooling liquid and a lid 123-2.

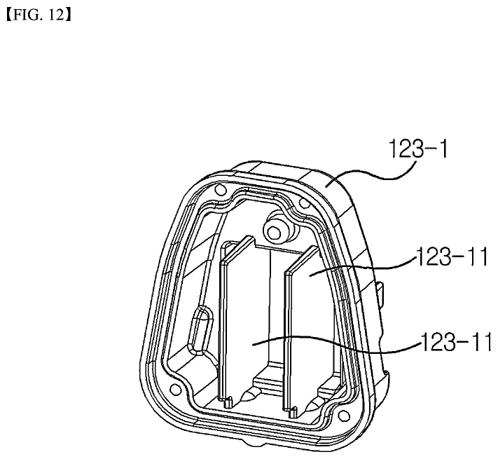

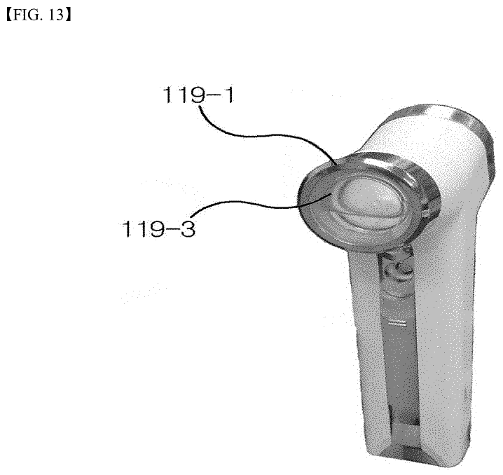

Furthermore, as illustrated in FIG. 12, an inner space of main body 123-1 may be formed with a plurality of lugs 123-11 to further increase the cooling efficiency.

In the given figure, the lid 123-2 of the second heat dissipation part 123 may be so formed as to be fixed to the main body 123-1 using a bolt and a screw, and an O-ring may be disposed for airtight-sealing between the main body 123-1 and the lid 123-2.

The controller 150 may perform an overall control of the multi-functional integrated skin beauty device 100 in response to an operation command inputted from the input part 160.

The methods for controlling the multi-functional integrated skin beauty device 100 using the controller 150 may be variably implemented, but the present invention is not particularly limited thereto.

For example, the controller 150 may allow mist to be spouted by driving the mist treatment part 110 in response to ON/OFF signals of mist function inputted from the input part 160, and may maintain the skin contact member 121 to be under or at an adequate temperature by driving the temperature stimulation treatment part 120 in response to cold/warm stimulation set-up signals.

Furthermore, the controller 150 may controllably allow generating an electric stimulation through the electroporation electrode by driving the EP treatment part 130 in response to ON/OFF signals of the electroporation function, and shedding an adequate light to be irradiated through the transparent window 141 by driving the light treatment part 140 in response to ON/OFF signals of light stimulation function.

The housing may be disposed with various display means in order to display the operations of the multi-functional integrated skin beauty device 100. For example, the housing may be disposed with LED lamps for displaying various types of information, including but not limited to, power ON/OFF state, operation state of mist treatment part 110, operation state of temperature stimulation treatment part 120, operation state of EP treatment part 130, operation state of light treatment part 140, and current cooling/warming set-up state.

The power supply part 170 may be so configured as to transform a DC power to an AC power for use in the multi-functional integrated skin beauty device 100, and may be so configured as to include a chargeable battery. The power supply part 170 may be variably configured to include a battery charging circuit as necessary.

Various other components including PCBs and batteries forming an electric circuit may be disposed inside an inner space of handle part 210.

The multi-functional integrated skin beauty device 100 may include a cradle part 230 performing a role of a resting stand or a mount.

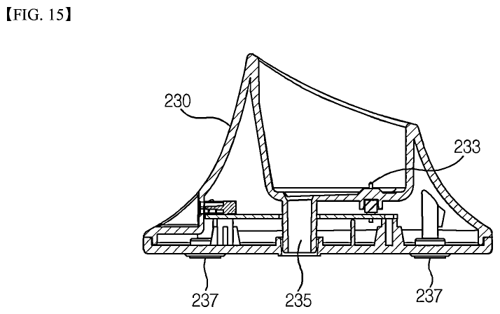

Referring to FIGS. 14 and 15, the cradle part 230 may be so configured as to allow a lower end of the handle part 210 to rest thereon, and the cradle part may be so disposed as to function as a battery for charging. At this time, a battery charging terminal 233 may be formed on the handle part 210 and the cradle part 230 for a mutual electric contact when the handle part 210 is mounted.

FIG. 14 illustrates a cradle part 230 viewed from above according to an exemplary embodiment, where a floor 231 of resting space of the cradle part 230 may be formed with a water drain hole 235 to allow the water flowing down through the handle part 210 to be drained out.

That is, the mists formed as water droplets in the course of being continuously spread may flow down along each portion of the handle part 210 including the mist function ON/OFF switch 119-2.

Thus, when the floor of the cradle part 230 is formed with a water drainage hole 235, water leaked through the handle part 210 may be drained out, whereby safety can be enhanced by preventing a short-circuit from being generated on the battery charging terminal 233.

Referring to FIG. 15, a portion formed with the water drain hole 235 at the cradle part 230 may be formed to be lower than a portion disposed with the battery charging terminal 233.

At this time, a descent slope may be formed at a predetermined angle from a portion where the battery charging terminal 233 is disposed to a portion where the water drainage hole 235 is formed.

Furthermore, a lower surface of cradle part 230 may be formed with a prop 237 and a water vessel (not shown) may be disposed to contain water drained through the water drainage hole 235.

While the present invention has been described with reference to the particular illustrative embodiments, it is not to be restricted by the embodiments but only by the appended claims. It is to be appreciated that those skilled in the art can change or modify the embodiments without departing from the scope and spirit of the present invention.

### DESCRIPTION OF REFERENCE NUMERALS

100: beauty device
110: mist treatment part
111: water tank
112: electrode module
113: ultrasonic oscillator
117: mist outlet
119-1: inlet cap
120: temperature stimulation treatment part
121: skin contact member
122: thermoelement (peltier device)
123: second heat dissipation part
123-1: main body
123-2: lid
124: first heat dissipation part
130: EP treatment part
132: outlying electrode member
140: optical treatment part
141: transparent window
150: controller
160: input part
170: power supply part
181: illumination part
210: handle part
220: head part
221: first head part
222: second head part
230: cradle part
233: battery charging terminal
235: water drainage hole
237: prop

What is claimed is:

1. A multi-functional integrated skin beauty device, the device comprising:
a mist treatment part generating sterilization water by electrolyzing water in a water tank and spraying the generated sterilization water in a mist shape;
a temperature stimulation treatment part applying a temperature stimulation to skin using an thermoelement;
a power supply part supplying an electric power;
an input part receiving an operation command; and
a controller controlling the mist treatment part and the temperature stimulation treatment part in response to the operation command inputted through the input part, wherein a housing forming an exterior appearance of the multi-functional integrated skin beauty device includes:
a pillar-type handle part graspable by a user with one hand; and
a first head part protruded from an upper end of the handle part toward a lateral surface, and a second protruded toward an opposite lateral surface thereof, and wherein the first head part is disposed with a raw water inlet to allow raw water to be poured into a water tank of the mist treatment part and the second head part is disposed with a skin contact member to allow applying a temperature stimulation by being contacted with skin.

2. The device of claim 1, further comprising:
an EP treatment part applying an electric signal to an EP electrode; and
a light treatment part outputting a light for skin care, wherein
the second head part is disposed with a transparent window formed along a surrounding of the skin contact member to output a skin care light, and wherein
the skin contact member and an outlying electrode member formed at a surrounding of the transparent window form the EP electrode, and wherein an uppermost end of the second head part is disposed higher than the first head part.

3. The device of claim 1, wherein the mist treatment part includes:
an electrode module disposed inside of the water tank to transform the raw water to sterilization water by electrolyzing the raw water;
an ultrasonic oscillator generating a fine particulate type mist by applying a vibration to the sterilization water generated by the electrode module; and
a mist outlet to allow spouting the mist generated by the ultrasonic oscillator therethrough, wherein
the water tank is formed of an approximately ‘¬’ shape across an upper space of the handle part from the raw water inlet, and wherein
at least a portion of the electrode module is so disposed at a position as to be visible through the raw water inlet.

4. The device of claim 3, wherein the mist outlet is disposed at an upper end of the handle part toward a direction formed with the raw water inlet, and the ultrasonic oscillator is disposed at a surrounding of the mist outlet, wherein a floor position of the water tank is so disposed as to be adjacent to a lowermost position of the ultrasonic oscillator.

5. The device of claim 1, further comprising:
an inlet cap configured to open/close the raw water inlet;
a visual effect plate configured to display a visual effect by being so disposed at an inside of the water tank as to allow being visible from the raw water inlet; and
an illumination part configured to shed an illumination light into an inside of the water tank from under the floor of the water tank, wherein
at least a portion of the water tank is so disposed with a transparent or translucent material as to allow the illumination light coming off from the illumination part to pass therethrough, and wherein
at least a portion of the inlet cap is so disposed as to allow an interior of the water tank to look enlarged.

6. The device of claim 1, further comprising: a cradle part so disposed with a battery charging terminal as to allow a lower end of the handle part to rest thereon, wherein the cradle part is disposed at a floor with a water drain hole to allow the water flowing down through the handle part to be drained out, and wherein a portion formed with the water drain hole is formed to be lower than a portion disposed with the battery charging terminal.
